(19) United States
(12) Patent Application Publication
(10) Pub. No.: US 2007/0249938 A1
(43) Pub. Date: Oct. 25, 2007

(54) SYSTEMS, DEVICES, AND METHODS EMPLOYING THERAPEUTIC ULTRASOUND OF LIVING TISSUES

(75) Inventor: Donald J. Shields, Arcadia, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Donald J. Shields, Arcadia, CA (US)

(21) Appl. No.: 11/621,072

(22) Filed: Jan. 8, 2007

Related U.S. Application Data
(63) Continuation-in-part of application No. 11/408,820, filed on Apr. 20, 2006.

(51) Int. Cl. A61B 8/00

(52) U.S. Cl. 600/447

(57) ABSTRACT

Systems, devices, and methods for delivering ultrasonic treatment to a subject. An ultrasound therapy device includes a waveform generator, one or more transducers, one or more sensors, and a controller. In some embodiments, the waveform generator is configured to generate a first driving signal having at least a first waveform segment and a second waveform segment different from the first waveform segment.

### CROSS-REFERENCE TO RELATED APPLICATION

This application is a continuation-in-part of U.S. patent application Ser. No. 11/408,820, filed Apr. 20, 2006, now pending, the contents of which are incorporated herein by reference in their entirety.

### BACKGROUND

1. Field

This disclosure generally relates to the field of therapeutic ultrasound and, more particularly, to systems, devices, and methods for providing in vivo ultrasound therapy to a subject.

2. Description of the Related Art

Humans and animals are composed of cells organized into various functional units or tissues, for example, bone, muscle, tendon, ligament, and cartilage. These and other commonly injured tissues are sometimes treated with therapeutic ultrasound.

Therapeutic ultrasound typically employs transducers to deliver ultrasound energy to the injured tissue. The thermal effects associated with this type of therapy can, however, damage the target tissue if transducer treatment elements are not kept in constant motion. Because of the risk of damaging target tissue, conventional ultrasound devices necessitate the use of trained and knowledgeable operators.

Research into the non-thermal effects of therapeutic ultrasound has uncovered other mechanisms of action, such as cavitation and acoustic streaming, which have been shown to cause the up-regulation of many cellular processes. These non-thermal effects of therapeutic ultrasound have been found to improve healing time and quality of healing in a wide variety of tissue types. For example, low-intensity therapeutic ultrasound has been shown to produce these same beneficial, non-thermal effects without creating an appreciable temperature increase in the treated tissues. Unlike higher intensity ultrasound therapy, low-intensity ultrasound treatment enables the static placement of the treatment element on the patient without thermally damaging the target tissue. Accordingly, these low-intensity devices are easy to use, thereby improving patient compliance.

The effects of therapeutic ultrasound on living tissues vary. For example, ultrasound typically has a greater affect on highly organized, structurally rigid tissues such as bone, tendons, ligaments, cartilage, and muscle. Increased healing of these tissues can be achieved with the application of either high-intensity therapeutic ultrasound or low-intensity therapeutic ultrasound. Due to their different depths within the body, however, the different tissue types require different ultrasonic frequencies for effective treatment. In addition, tissues respond to ultrasound in different ways depending on the chronicity of the injury to the tissue. Accordingly, acute and chronic injuries are treated differently.

Utilizing these scientific principles the above-mentioned apparatuses and methods use ultrasonic energy for in vivo therapeutic treatment of bone tissue with carrier frequencies and therapeutic ultrasound pulses. Typical apparatuses often allow for the selection of certain treatment parameters such as ultrasound frequency, pulse intensity, etc., but typically produce a single, fixed waveform during each treatment application. Moreover, the typical ultrasound apparatuses are designed to treat a single type of tissue with a single, specific and fixed ultrasonic frequency, pulse intensity, pulse ratio, pulse duration, and pulse repetition rate during each therapeutic use. Because these apparatuses generally employ singular waveforms, the transducer treatment elements usually require substantial movement about a treatment area to avoid thermally damaging the target tissue.

The present disclosure is directed to overcome one or more of the shortcomings set forth above, and provide further related advantages.

### BRIEF SUMMARY

In one aspect, the present disclosure is directed to an ultrasound therapy device for delivering ultrasonic treatment to a biological entity. The ultrasound therapy device includes a waveform generator, one or more transducers, one or more sensors, and a controller.

The waveform generator is configured to generate a first driving signal having at least a first waveform segment and a second waveform different from the first waveform segment. The one or more transducers are electrically coupleable to the waveform generator and are configured to receive the first driving signal and to generate a first ultrasonic signal based in part on the first driving signal. The one or more sensors are operable to determine at least one physiological characteristic of the biological entity. The controller is electrically coupled to the waveform generator and the one or more sensors. In some embodiments, the controller is configured to perform a comparison of the determined at least one physiological characteristic of the biological entity to stored reference data, and to generate a response based in part on the comparison.

In another aspect, the present disclosure is directed to a method of treating at least one condition associated with injured tissue in a subject.

The method includes contacting a location on a biological interface of the subject with an ultrasound delivery device comprising one or more ultrasound transducers. The one or more ultrasound transducers are operable for providing an ultrasonic signal comprising at least a first waveform segment and a second waveform segment, the first waveform segment having at least one of an intensity, frequency, pulse intensity, pulse duration, pulse ratio, or pulse repetition rate different from the second segment.

The method further includes applying a sufficient amount of current to emit a therapeutically effective amount of ultrasonic energy from the ultrasound delivery device.

In another aspect, the present disclosure is directed to an ultrasound therapy system for delivering ultrasonic treatment to a biological entity. The system includes an inductive power supply and an ultrasound delivery device.

The inductive power supply includes a primary winding and is operable to produce a varying magnetic field. The ultrasound delivery device includes a waveform generator, a secondary winding, and one or more transducers. The waveform generator is configured to generate a first driving signal having at least a first waveform segment and a second waveform segment, the second waveform segment different from the first. The secondary winding is electrically coupled to the waveform generator and is operable for providing a potential to the waveform generator in response to a varying electromagnetic field applied to the secondary winding. The one or more transducers are electrically coupled to the waveform generator, and are configured to receive the first driving signal and to generate a first ultrasonic signal based in part on the first driving signal.

In yet another aspect, the present disclosure is directed to an article of manufacture for delivering ultrasound therapy. The article of manufacture includes an ultrasound therapy device for delivering ultrasonic treatment to a biological entity, and a package insert.

The ultrasound therapy device includes a waveform generator, one or more transducers, one or more sensors, and a controller. The waveform generator is configured to generate a first driving signal comprising at least a first waveform segment and a second waveform segment different from the first waveform segment. The one or more transducers are electrically coupled to the waveform generator and are configured to receive the first driving signal and to generate a first ultrasonic signal based in part on the first driving signal. The one or more sensors are operable to determine at least one physiological characteristic of the biological entity. The controller is electrically coupled to the waveform generator and the one or more sensors. In some embodiments, The controller is configured to perform a comparison of the determined at least one physiological characteristic of a biological entity to stored reference data, and to generate a response based in part on the comparison.

The package insert provides instructions for administering, to a subject in need of ultrasonic therapy, a therapeutically effective amount of at least one dosage form of ultrasonic energy.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top front view of an exemplary ultrasound therapy system for delivering ultrasonic treatment to a biological entity according to one illustrated embodiment.

FIG. 1B is a top plan view of an exemplary ultrasound therapy system for delivering ultrasonic treatment to a biological entity according to another illustrated embodiment.

FIG. 2 is a schematic diagram of an ultrasound therapy device according to one illustrated embodiment.

FIGS. 3A and 3B are time versus amplitude plots of exemplary uni-variant and multi-variant waveforms according to multiple illustrated embodiments.

FIG. 4 is a schematic diagram of an ultrasound therapy device according to one illustrated embodiment.

FIG. 5A is a schematic diagram of an ultrasound therapy device in the form of one transducer according to one illustrated embodiment.

FIG. 5B is a schematic diagram of an ultrasound therapy device in the form of a plurality of transducers according to another illustrated embodiment.

FIGS. 6A, 6B and 6C are top plan views of an ultrasound therapy system according to multiple illustrated embodiments.

FIGS. 7A, 7B and 7C are schematic diagrams of an ultrasound therapy device according to one illustrated embodiment.

FIG. 8 is a schematic diagram of an inductively power ultrasound therapy device according to one illustrated embodiment.

FIG. 9 is a flow diagram of a method of treating at least one condition associated with injured tissue in a subject according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with ultrasound devices including, but not limited to, voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an ultrasound therapy device including “a transducer” includes a single transducer, or two or more transducers. It should also be noted that the term “or” is generally employed in its sense including “and/or” unless the content clearly dictates otherwise.

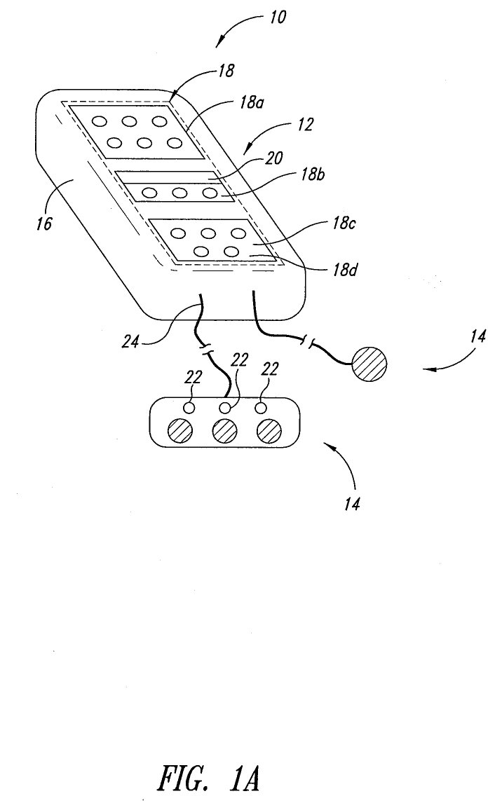

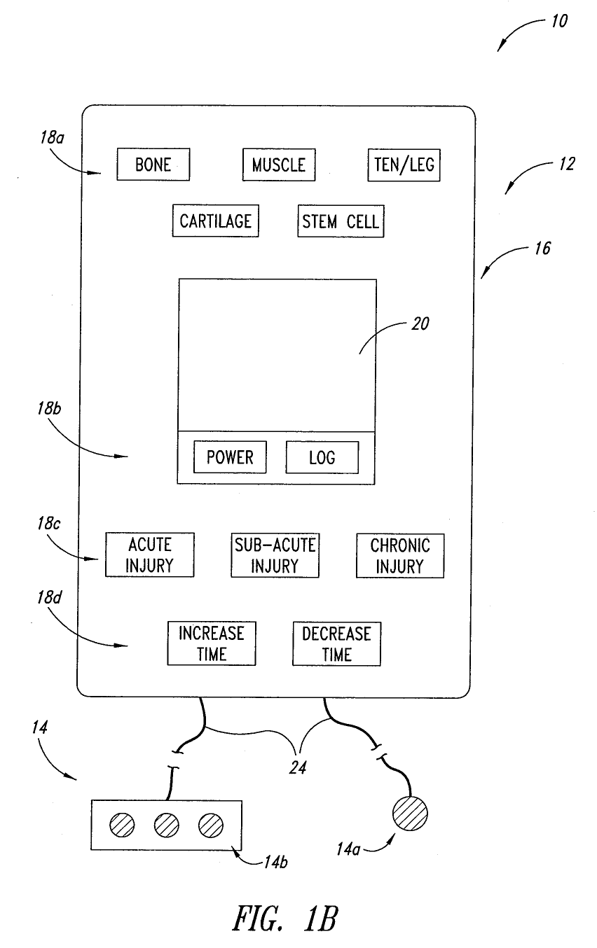

FIGS. 1A and 1B show an exemplary ultrasound therapy system 10 for delivering ultrasonic treatment to a biological entity according to one illustrated embodiment. The system 10 includes an ultrasound therapy device 12 including a transducer 14. The transducer 14 may take the form of a single transducer 14a, a plurality of transducers (a three transducer set up is depicted in 14b), and/or one or more arrays of transducers. In some embodiments, the ultrasound therapy device 12 may include at least a single transducer 14a and plurality of transducers 14b.

The system 10 may be operable to simultaneously utilize multiple transducer treatment elements. In some embodiments, the system 10 may include multiple drive circuits (e.g., one drive circuit for each transducer 14) and may be configured to generate varying waveforms from each connected transducer (e.g., multiple waveform generators, and the like).

The system 10 may further include a housing 16, a control module 18, and a display 20. The system 10 may also include one or more sensors 22 operable to determine at least one physiological characteristic of a biological entity. Examples of a physiological characteristic include, for example, an impedance, a temperature, a density, a vital statistic (e.g., a blood pressure, a pulse), and/or a fat content, and the like. Other physiological characteristics can also be determined.

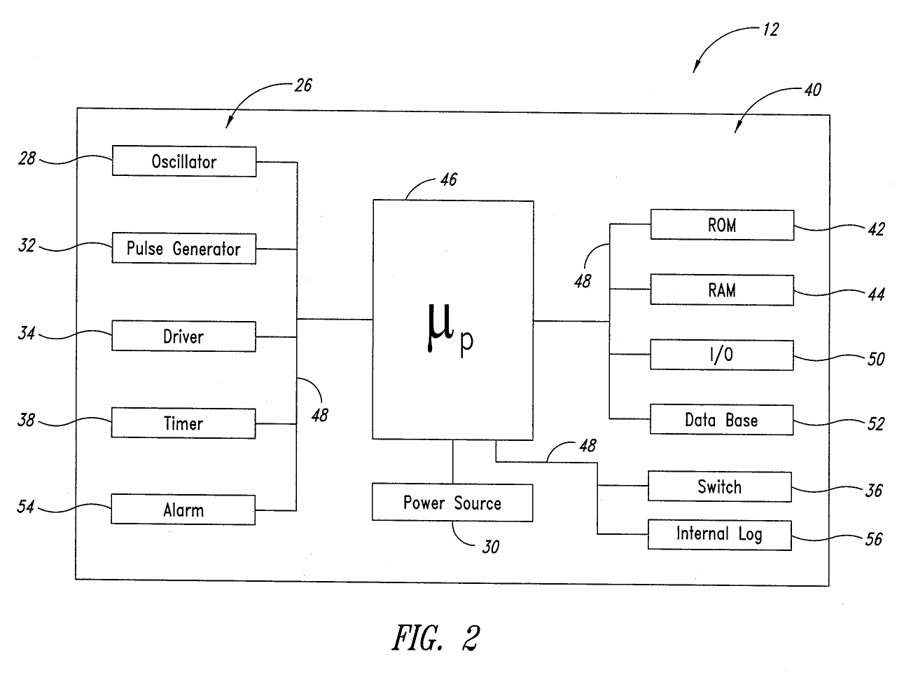

As shown in FIG. 2, the ultrasound therapy device 12 may further include a waveform generator 26, a controller system 40, and a power supply 30.

The waveform generator 26 may include an oscillator 28 and a pulse generator 32 operable to generate a first driving signal. In some embodiments, the oscillator 28 takes the form of a radio frequency (RF) oscillator operable to provide an RF signal to the transducer 14 causing the transducer 14 to ultrasonically vibrate and generate ultrasonic energy. The ultrasonic energy is subsequently transmitted to the injured tissue.

In some embodiments, the waveform generator 26 may be configured to generate the first driving signal based in part on a user input. The user input may include, for example, at least one of an intensity, a frequency, a pulse ratio, a pulse intensity, a pulse duration, a pulse frequency, a pulse repetition rate, a continuous waveform frequency, a continuous waveform intensity, a treatment type, a treatment time, a treatment duration, a treatment time increase or decrease, a treatment interval rate, a lesion depth, a degree of tissue injury, a tissue type, and the like, or combinations thereof. The waveform generator 26 may further include a single oscillator 28 (e.g., variable frequency RF) programmed to deliver one or more frequencies based in part on the user input. In some other embodiments, the waveform generator 26 may include a plurality of oscillators 28, one for each type of tissue. In such embodiments, each of the oscillators 28 is pre-programmable to transmit a specific signal based in part on the tissue type associated with the particular oscillator 28. The oscillator 28 is operable to generate a signal with a frequency ranging from about 20 kHz to about 3 MHz.

The pulse generator 32 may generate pulsed periods and non-pulsed (or inactive) periods. A pulse ratio refers to the ratio of time that the pulse generator 32 is active (generating pulsed periods) to the time that the pulse generator 32 is inactive (generating non-pulsed periods). Similarly, a duty cycle refers to a ratio of a pulse signal duration relative to a pulse signal period. For example, a pulse signal duration of 10 μs and a pulse signal period of 20 μs, correspons to a duty cycle of 0.5. In some embodiments, the pulse generator 32 generates pulsed and non-pulsed periods in a pulse ratio dependent on the type of injury. In general, however, the pulse ratio ranges from about 1:1 to about 1:20. In some embodiments, the pulse ratio ranges from about 1:1 to about 1:8. In one exemplary embodiment, for chronic injuries, the pulse ratio ranges from about 1:1 to about 1:2, and for acute injuries, the pulse ratio ranges from about 1:3 to about 1:4. Other pulse ratios may also be possible.

The pulse generator 32 varies the intensity of the pulses depending, in part, on the type of tissue and injury selected. For example, for chronic injuries, the pulse generator 32 outputs a pulse having a greater intensity than the pulse output for acute injuries. Although dependent on the type of injury and tissue, the pulse generator 32 is generally operable to vary the intensity of a pulse from about 20 mW/cm² to about 3 W/cm².

In some embodiments, one or more continuous waveform segments may be combined with the generated pulsed periods and/or non-pulsed (or inactive) periods.

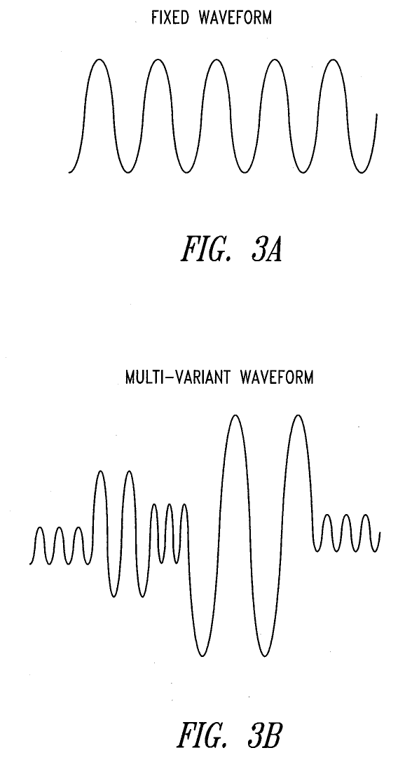

Ultrasound therapy may be provided in one or more treatment segments. In some embodiments, the waveform of each treatment segment may differ from the preceding or subsequent waveform segment in at least one waveform characteristic (e.g., intensity, frequency, pulse intensity, pulse duration, pulse ratio, pulse repetition rate, and the like). Waveforms having segments that vary in only one characteristic are generally said to be uni-variant (see e.g., FIG. 3A), and waveforms having segments that vary in more than one characteristic are generally said to be multi-variant (see e.g., FIG. 3B). Waveforms with constant amplitude and frequency are generally said to be continuous waveforms. Continuous waveforms may vary in intensity and/or frequency.

During treatment, the ultrasound device 12 may cycle through the selected waveform segments at selected times to treat the selected tissue type until all the waveforms are used and/or the treatment time elapses. This cycling of varying waveform segments enables the safe application of therapeutic ultrasound through the generally static placement of transducer treatment elements. The varying waveform modulates and controls the power density received by the target tissue throughout the treatment and maintains the power density within optimal therapeutic levels. In some embodiments, the ultrasound device 12 may cycle through one or more uni-variant, multi-variant, and continuous waveforms, or combinations thereof.

In some embodiments, the pulse generator 32 may vary the intensity of a single pulse or a series of pulses throughout a single treatment session. For example, the pulse generator 32 may vary the intensity of a single pulse in a series of pulses delivered in a single treatment session such that each pulse outputted by the pulse generator 32 has a multi-variant waveform. The pulse generator 32 may also vary the intensity of the series of pulses, such that each pulse in the series of pulses delivered in a single treatment session has a uni-variant waveform. The pulse generator 32 may be operable to vary the intensity of each pulse and the intensity of the series of pulses delivered in a single treatment session.

The generated pulses may take a variety of forms including multi-variant waveforms, uni-variant waveforms, continuous waveforms, or combinations thereof. In some embodiments, one or more pulses in a pulse series may have uni-variant waveforms and one or more pulses in the pulse series may have multi-variant waveforms. In some further embodiments, the generated pulses may include one or more continuous waveforms segments.

The frequency needed to reach each tissue type is generally known in the relevant art. The pulse generator 32 may control the intensity, frequency, pulse intensity, duration, ratio, and/or repetition rate, each of which is determined based in part on the injury type and the tissue type.

Depending on the injury type, the pulse generator 32 generally generates a pulse having a pulse duration ranging from about 10 μs to about 2,500 μs, a pulse repetition rate (frequency) ranging from about 50 Hz to about 10,000 Hz and a pulse intensity ranging from about 20 mW/cm² to about 3 W/cm². In one exemplary embodiment, the pulse frequency is about 1,000 Hz and the pulse duration ranges from about 100 μs to about 400 μs.

In some embodiments, the ultrasound therapy device 12 may include a waveform generator 26 configured to generate a first driving signal including at least a first waveform segment, and a second waveform segment different from the first segment. Depending on the input (e.g., the injury type, the lesion depth, the degree of tissue injury, the tissue type, and the like) the waveform generator 26 may generate a first signal having varying characteristics including, for example, the length of each waveform segment. In some embodiments, the waveform generator 26 is operable to generate continuous waveforms, pulsed waveforms, or combinations thereof. For example, in some embodiments, the waveform generator 26 may generate a first signal having varying characteristics including, for example, varying intensities, frequencies, pulse intensities, pulse durations, pulse ratios, and/or pulse repetition rates for each waveform segment, based in part on the input. In some embodiments, the first waveform segment has at least one of an intensity, a frequency, a pulse intensity, a pulse duration, a pulse frequency, a pulse ratio, or a pulse repetition rate different from the second waveform segment. In some embodiments, the first and the second waveform segments are each independently selected from one or more single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, or combinations thereof including, for example, single and multi-frequency formed waves. In some embodiments, the ultrasound therapy device 12 may further be configured to cycle through the at least first and second waveform segments for a limited treatment time. In some embodiments, the generated first ultrasonic signal compromise at least one of a continuous or a pulsed ultrasonic treatment wave, or combinations thereof.

In some embodiments, the intensity of the first ultrasonic signal ranges from about 0.02 W/cm² to about 3 W/cm². In some embodiments, the intensity of the first ultrasonic signal ranges from about 0.02 W/cm² to about 1.5 W/cm². In some embodiments, the intensity of the first ultrasonic signal ranges from about 0.4 W/cm² to about 1.5 W/cm². In some embodiments, the pulse ratio of the first ultrasonic signal ranges from about to about 1:1 to about 1:20. In some embodiments, the frequency of the first ultrasonic signal ranges from about 0.05 MHz to about 3 MHz. In some embodiments, the pulse repetition rate of the first ultrasonic signal ranges from about 500 Hz to about 2500 Hz. In some embodiments, the pulse repetition rate of the first ultrasonic signal ranges from about 50 KHz to about 10,000 Hz. In some embodiments, the pulse duration of the first ultrasonic signal ranges from about 10 μs to about 2,500 μs. In some embodiments, the first ultrasonic signal comprises a pulse duration ranging from about 10 μs to about 2,500 μs, a pulse ratio ranging from about 1:1 to about 1:8, a pulse repetition rate ranging from about 50 Hz to about 10,000 Hz, and a pulse intensity ranging about 0.02 W/cm² to about 3 W/cm². In some embodiments, the first ultrasonic signal comprises a pulse repetition rate ranging from about 500 Hz to about 2,500 Hz, and a pulse duration ranging from about 100 μs to about 500 μs.

Referring to FIG. 2, the control system 22 may include one or more controllers 46 such as a microprocessor, a digital signal processor (DSP) (not shown), an application-specific integrated circuit (ASIC) (not shown), and the like.

The device 12 may also include a control system 40 for selectively controlling various aspects of the ultrasound therapy device 12. The control system 40 may include one or more memories, for example, read-only memory (ROM) 42 random access memory (RAM) 44, and the like, coupled to the controller 46 by one or more busses 48. The control system 40 may further include one or more input devices 50 including, for example a display 20, a controller module 18 including one or more treatment controller modules 18a, 18b, 18c, 18d, and the like, or any peripheral device. In some embodiments, the controller 46 is configured to compare a physiological characteristic of a biological entity to a database 52 of stored reference values, and to generate a response based in part on the comparison. For example, the controller 46 may be configured to compare a measured impedance, temperature, density, or fat content of a biological entity to a database 52 of stored reference values, and to generate a response based in part on the comparison. The database 52 of stored values may include characteristic physiological data including, for example, characteristic impedance data, characteristic temperature data, characteristic density data, characteristic fat content data, characteristic treatment delivery data, and the like. In some embodiments, the generated response includes at least one of a response signal, a comparison plot, a treatment code, a diagnostic code, a test code, an alarm, a change to a treatment parameter, a response signal operable to terminate the first driving signal, and the like.

As shown in FIG. 4, the waveform generator 26 may be electrically connected to a driver 34 operable to modulate the output of the oscillator 28 in the form of an RF oscillator with the signal generated by the pulse generator 32 to generate a single signal. In one exemplary embodiment, the driver 34 generates a signal having a frequency ranging from about 50 kHz to about 3 MHz during pulsed periods. In some embodiments, the driver 34 also amplifies the resulting signal in order to deliver power having a maximum intensity for safe and effective ultrasonic therapy. In some embodiments, the driver 34 is operable to generate a signal selected from one or more single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, or combinations thereof, including single and multi-frequency formed waves. In one exemplary embodiment, the power has a maximum intensity of about 3 W/cm². In another exemplary embodiment, the driver 34 is operable to deliver power having an intensity ranging from about 30 mW/cm² to about 500 mW/cm².

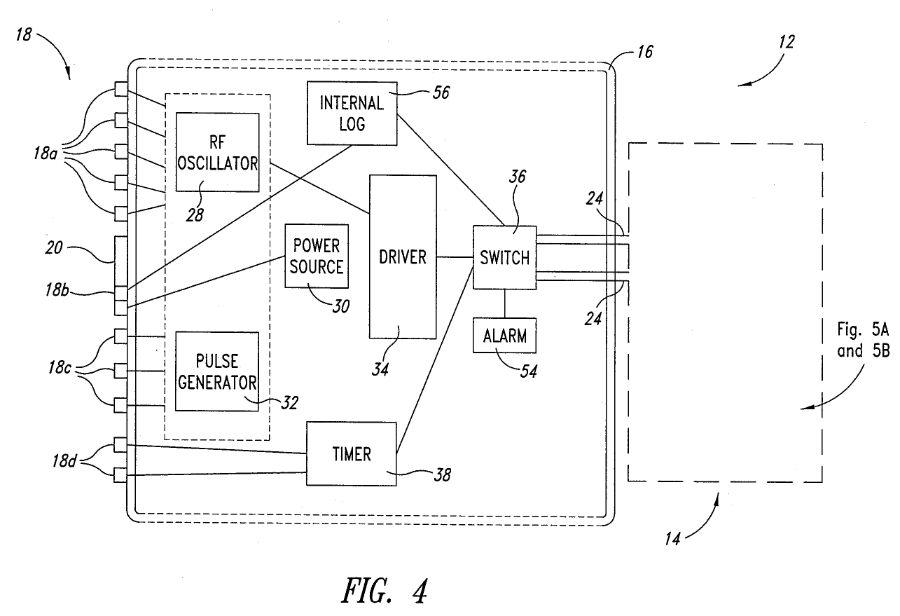

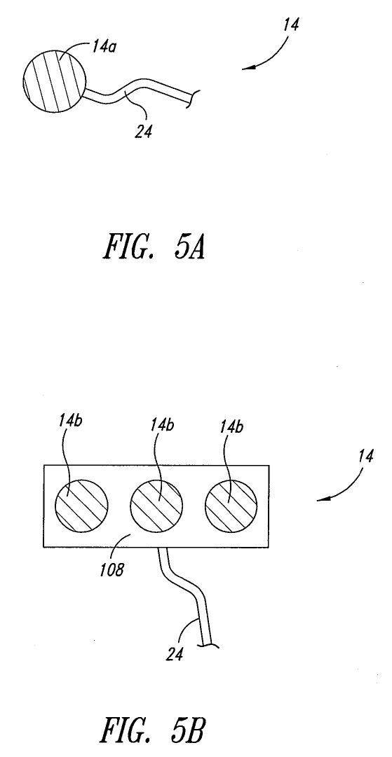

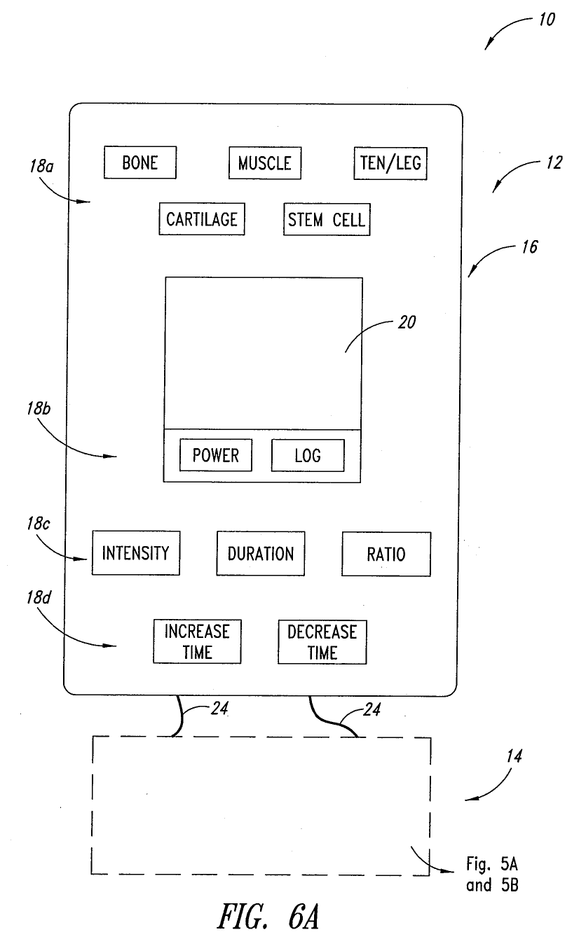

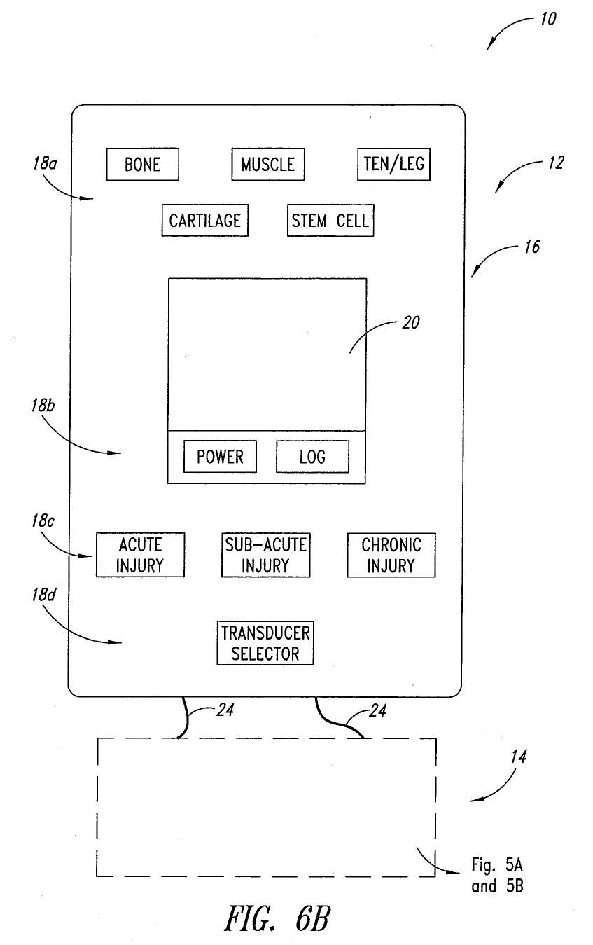

In some embodiments, the driver 34 is electrically coupled to a switch 36. The switch 36 is electrically coupled with one or more transducer cables 24, which electrically communicates with the transducer 14. In another exemplary embodiment, multiple cables 24 electrically communicate with the transducer 14. In use, the switch 36 receives a signal from the driver 34 and transmits it via cable 24 to the transducer 14. Referring to FIGS. 5A and 5B, the transducer 14 may be a single transducer (as shown, for example, in FIG. 5A), a plurality of transducers, and/or an array of transducers (as shown, for example, in FIG. 5B).

The ultrasound therapy device 12 can further include a timer 38 for timing the treatment. As shown in FIG. 4, an input device element (e.g., one or more selectors, buttons, and the like) of the control module 18 is electrically connected to the timer 38 for setting the treatment time. The input device element can have any construction suitable for setting the treatment time. For example, the input device element may comprise a button that alters the treatment time upon depression. Additionally, the housing 16 may include a touch-sensitive screen having areas designated for time selection, for altering the treatment time upon touching the desired area of the screen, and the like. The input device element may be capable of scrolling, rotating, or otherwise altering the treatment time. In one exemplary embodiment, the treatment time ranges from about 5 minutes to about 60 minutes per session and from 1 to 3 sessions per 24-hour period. In another exemplary embodiment, the treatment time ranges from about 30 minutes to about 45 minutes per session. Daily treatments may continue until the injured tissue is partially or completely healed.

The timer 38 is electrically connected to the switch 36. Upon setting the timer 38 and starting a treatment session, the switch 36 transmits the signal from the driver 34 to the cable 24. When the treatment time has elapsed, the switch 36 ceases to deliver the signal from the driver 34 to the cable 24, thereby ending delivery of ultrasonic energy. In one embodiment, the timer is pre-programmed with treatment times based in part on tissue and injury types. The timer may also include an override feature operable for setting treatment times outside the preset parameters.

In some embodiments, a controller 46, in the form of a microprocessor, associated with the control module 18 may determine in part the order in which the device 12 cycles through each treatment segment. The microprocessor may be factory pre-programmed or user accessible via a USB connection or direct access from the device controls via a, for example, touch screen, rotating dial, and/or button push.

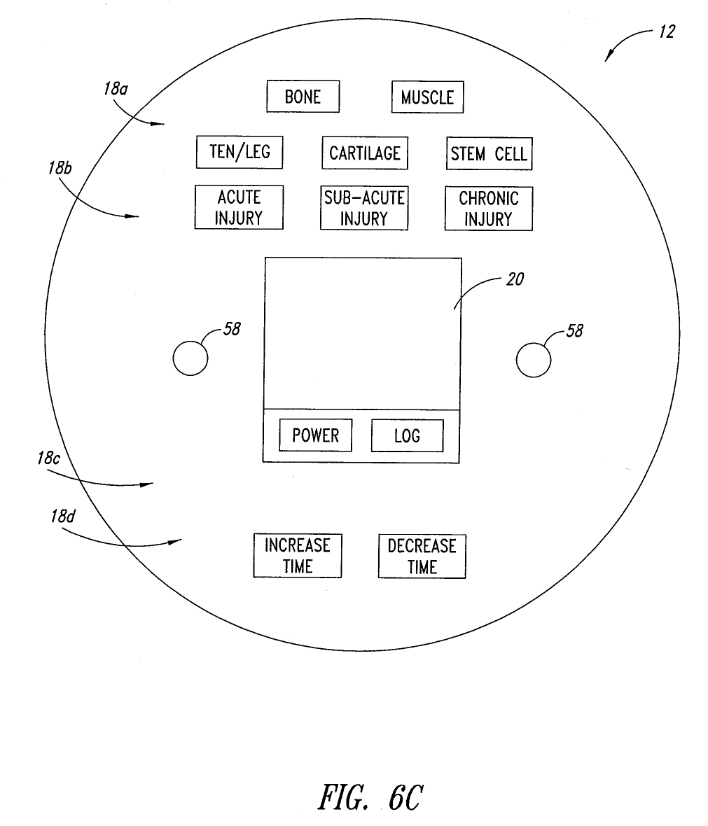

As shown in FIGS. 6A, 6B, and 6C the ultrasound therapy device 12 may include control module 18 including one or more treatment control modules 18a, 18b, 18c, 18d each including one or more input device elements for providing one or more treatment parameters. Examples of treatment parameters include, without limitation, a treatment type, a treatment time, a treatment duration, a lesion depth, a degree of tissue injury, a tissue type, an intensity, a frequency, a pulse ratio, a pulse intensity, a pulse duration, a pulse frequency, a pulse repetition rate, and the like.

The one or more treatment control modules 18a, 18b, 18c, 18d can generally be similar, or different and my have any construction suitable for providing input and/or output. The one or more treatment control modules 18a, 18b, 18c, 18d may also be operable to effect a user or treatment selection. For example, each treatment control module 18a, 18b, 18c, 18d may comprise an input device element in the form of a button for effecting a selection upon depression and or an output device in the form of a screen or a touch-sensitive screen having areas designated for input and/or output. For example, the housing 16 may include a touch-sensitive screen 20 having areas designated for inputting and/or outputting treatment control parameters and that, for example, effect selection or treatment input upon touching a desired area of the screen. The control module 18 may take the form of a single control module capable of scrolling, rotating or otherwise effecting selection and/or providing input/output capabilities. In one exemplary embodiment, the one or more treatment control modules 18a, 18b, 18c, 18d of control model 18 are adapted to enable input of tissue type, and/or degree of injury. The tissue type, as well as the depth at which the tissue lies within the body, is used to determine, in part, the ultrasonic frequency of the pulses generated by the device 12.

The control module 18 may be used to input or provide treatment parameters. For example, based on selections, pre-programmed instruction, and/or inputs made by activating or engaging one or more input device elements (e.g., a treatment selection button, a touch-screen area, and the like), a waveform (e.g., a first driving signal, and the like) is generated for providing treatment. The waveform generated may comprise a plurality of waveform segments, each segment having at least one characteristic different from the previous or subsequent waveform segment. In some embodiments, the different waveform segments may differ in one or more characteristics such as intensity, frequency, pulse ratio, pulse intensity, pulse duration, pulse repetition rate, and the like. In some embodiments, the ultrasound therapy device 12 is operable to cycle through the selected waveform segments. Each waveform segment is activated for a certain period of time before the device 12 cycles to the next waveform segment. The device 12 may continue to cycle through the waveform segments until all the segments have been used at least once, and/or the total treatment time elapses.

In some embodiments, the control module 18 may be pre-programmed to deliver waveform characteristics based on the type of injury inputted, selected, preprogrammed, and/or provided. In one embodiment, the control module 18 may be pre-programmed by a manufacturer to enable and/or deliver a specific waveform based on the tissue selected. In some embodiments, one, some, or all of the treatment control modules 18a, 18b, 18c, and 18d may be included. The pre-programmed treatment control module may include variations in waveform characteristics such as the length of each treatment segment and the cycling order of the treatment segments. Each treatment control module 18a, 18b, 18c, 18d may communicate with the waveform generator 26. In some embodiments, the control module 18 may include at least three input device elements, one each for chronic injuries, sub-acute injuries, and acute injuries. In some embodiments, the control module 18 includes one or more input device elements, one for each tissue type including bone, muscle, cartilage, tendons and/or ligaments, stem cells, and the like.

In some embodiments, a first input device element can control the pulse intensity, a second input device element can control the pulse duration, and a third input device element can control the pulse ratio. The combination of pulse duration and pulse ratio generates the pulse repetition rate. As such, the pulse repetition rate is automatically generated by the selection of a pulse duration and a pulse ratio. Because this fine-tuning of treatment variables requires extensive knowledge of therapeutic principles and medical practices, certain embodiment are designed for use by, for example, qualified operators.

An ultrasonic frequency can be first selected by tissue type through a plurality of input device elements. Pulse intensity may then be selected, since the selection of pulse intensity affects the available parameters for the remaining waveform characteristics. The input device elements can be pre-programmed to prevent selection of combinations of waveform characteristics and treatment segment times that may lead to thermal or other tissue damage. Once pulse intensity is selected, the pulse duration is selected. Next, the pulse ratio is selected, and finally, the treatment time is set. After setting the first waveform, the user may set another waveform, or simply begin treatment with a single waveform.

The device 12 can also include an alarm 54 for alerting the user of an event. For example, the alarm 54 can indicate a completion of a treatment portion, treatment completion, elapse of a treatment time, a malfunction, an error, and the like. The alarm 54 may be electrically connected to the switch 36 and configured to ceases deliver of the first driving signal from the driver 34 to the cable 24, and instead energize the alarm 54, which alerts the user to, for example, the completion of the treatment. The alarm 54 may alert the user by, for example, an audible alarm that rings or otherwise makes a noise indicating the completion of the treatment, and the like. Alternatively, the alarm may alert the user by flashing lights, and or displaying a code, a message, an instruction, and the like.

The switch 36 may also comprise an interrupt feature for pausing treatment, for example, either when a loss of contact between the transducer 14 and the treatment area occurs or when the transducer 14 is otherwise not functioning properly. When such an event takes place, the switch 36 will cease delivering the signal from the driver 34 to the transducer 14, and will energize the alarm 54 to alert the user to the malfunction.

The device 12 may further include at least one display screen 20 and an internal log 56 for documenting and/or tracking a treatment variable including, for example, a usage of the device 12 and treatment specifics. In addition, the device 12 may be operable for allowing the entering of, for example, patient data. For example, the control module 18 may include a input screen, a keyboard or the like for entering patient data, such as the patient's name, age, weight, injury complained of, and the like. The display screen 20 can be any suitable screen for displaying and/or inputting the desired information, for example a liquid crystal display screen. Additionally, at least two display screens 20 can be provided, one for displaying information related to treatment specifics, and one displaying elapsed time during treatment.

The internal log 56 can comprise any suitable mechanism, such as a controller in the form of a microprocessor, and can be accessed by a log input device element located on the device 12. For example, when accessed, the log information may appear on the at least one display screen 20. The internal log 56 may track, and/or store information such as the number of treatments performed, the length of each treatment, the date and time each treatment was performed, the types of tissues and/or injuries treated, and the like. In some embodiments, the internal log 56 may take the form of one or more microprocessors and/or memories.

The internal log 56 is connected to the switch 36 and the timer 38. In use, the switch 36 delivers information to the internal log 56 that then stores the received information. Additionally, the internal log 56 may store the timing information received from the timer 38. As noted above, the stored information can later be accessed via a keyboard, a touch-screen menu, through sequential depressions of the log button, and the like, and the information is displayed on the display screen 20 for analysis by the user.

The transducer cable 24 may be connected to the transducer 14 electrically, wirelessly, optically, by RF, by fiber optics, and the like. The transducer 14 may be a single transducer (as shown, for example, in FIG. 5A), a plurality of transducers, and/or an array of transducers (as shown, for example, in FIG. 5B). In addition, multiple transducers or transducer arrays may be connected through multiple cables 24 to a single device 12. Transducer arrays and multiple transducers are used when the treatment area is relatively large, and/or when there are multiple treatment areas.

In one embodiment, the one or more transducers 14 take the form of a combined frequency output transducer having multiple fixed or variable frequency transducer elements. A plurality of transducers may be used to provide simultaneous therapy to various depths of thick target tissues such as inflamed joint capsules or muscle tissue.

In one exemplary embodiment, the transducer 14 may include a single transducer element or a plurality of transducer elements, and may take the form of any conventional transducer, such as those made of piezoelectric materials. In some embodiments, each transducer may comprise a generally round disc approximately 1 inch in diameter including a treatment surface 15 and a visible surface (not shown). Although described and illustrated as a generally round disc, it is understood that the transducer can have any other suitable geometric shape, and/or form.

The transducer 14 may further comprise one or more sensor 22 in the form of, for example, a temperature sensor for sensing the temperature of the patient's skin, target tissue within the body, and/or transducer treatment element during treatment. The at least one temperature sensor is electrically connectable to the switch 36, which is configured to cease delivery of waveform information to the transducer if the temperature of the patient's skin reaches a safety, threshold level, and/or predetermined value. The transducer 14 may further comprise a component physically coupled to the visible surface of the transducer 14 to alert the user to a malfunction. For example, the transducer 14 may include at least one light emitting diode (LED) 58 on the visible surface of the transducer 14, which lights up or flashes when a malfunction occurs. An LED 58 may be associated with a sensor 22 (e.g., a temperature sensor, and the like) can be placed on the control module or on both the control module 18 and the transducer treatment element. Instead of, or in addition to the LED 58, the transducer 14 may include an audible alarm 54 that alerts the user to the malfunction.

The device 12 may further include a powered source 30. In some embodiments, the ultrasound therapy device 12 may include a rechargeable power source in the form of at least one of a button cell, a chemical battery cell, a fuel cell, a secondary cell, a lithium ion cell, a nickel metal hydride cell, a super-capacitor, a thin film secondary cell, an ultra-capacitor, a zinc air cell, and the like. In one embodiment, for example, the ultrasound therapy device 12 includes an internal battery pack sufficient to power all elements of the device 12. In some embodiments, the device 12 may be powered by plugging it into an electrical socket.

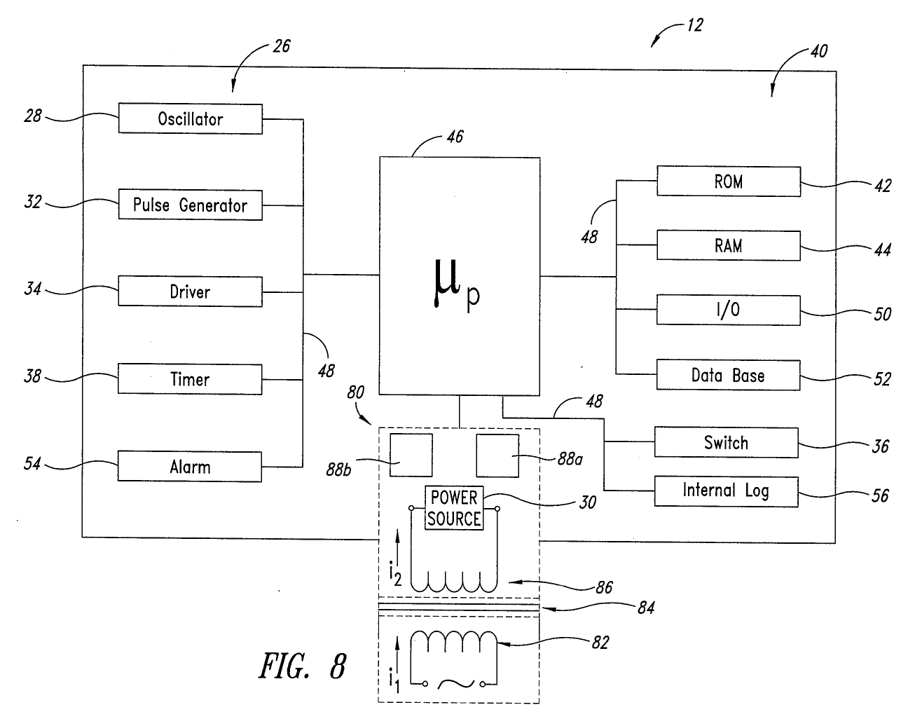

As shown in FIG. 8, the ultrasound therapy device 12 may include an inductive power supply system 80 including a primary winding 82 operable to produce a varying magnetic field 84, and a secondary winding 86 electrically coupled to the waveform generator 26 and operable for providing a potential to the waveform generator 26 in response to a varying electromagnetic field applied to the secondary winding 86. The inductive power supply system 80 may also include discrete and/or integrated circuit elements 88a, 88b to control the voltage, current and/or power delivered to the ultrasound therapy device 12.

The inductive power supply 4 is operable to transfer energy, via inductive coupling, from one component to another through a shared magnetic field 3. A change in current flow (i₁) through one component may induce a current flow (i₂) in the other component. The transfer of energy results in part from the mutual inductance between the components. For example, the inductive power supply 4 is operable to transfer energy, via inductive coupling, from a primary winding 82 to a secondary winding 86 through a shared magnetic field 84.

The windings 82, 86 may include one or more complete turns of a conductive material in a coil, and may comprise one or more layers. Examples of suitable conductive materials include conductive polymers, metallic materials, copper, gold, silver, copper coated with silver or tin, aluminum, and/or alloys. In some embodiments, the windings 82, 86 may comprise, for example, solid wires, including, for example, flat wires, strands, twisted strands, sheets, and the like. Examples of primary windings 82 include a coil, a winding, a primary coil, a primary winding, an inductive coil, a primary inductor, and the like. Examples of secondary windings 86 include a coil, a winding, a secondary coil, a secondary winding, an inductive coil, a secondary inductor, and the like. The secondary windings may include one or more complete turns of a conductive material in a coil, and may comprise one or more layers. The inductive power supply may be operable to provide at least one of an alternating current or a pulsed direct current to the primary winding 82. In some embodiments, the ultrasound therapy device 12 includes a rechargeable power source 88 electrically coupled to the waveform generator 26, and electrically coupled in parallel with the secondary winding 86 to receive a charge thereby. In some embodiments, the rechargeable power 88 source sinks and sources voltage to maintain a steady state operation of the ultrasound device 12.

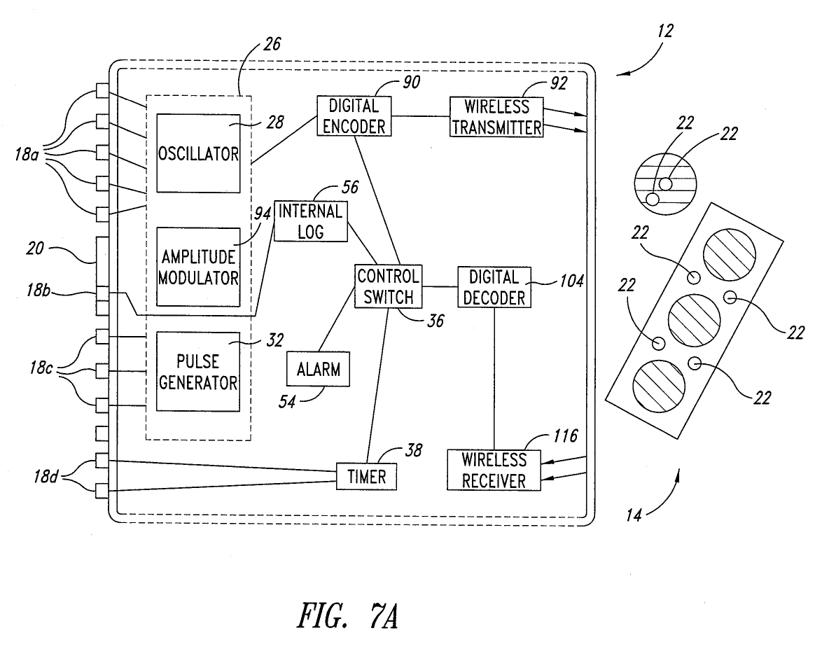

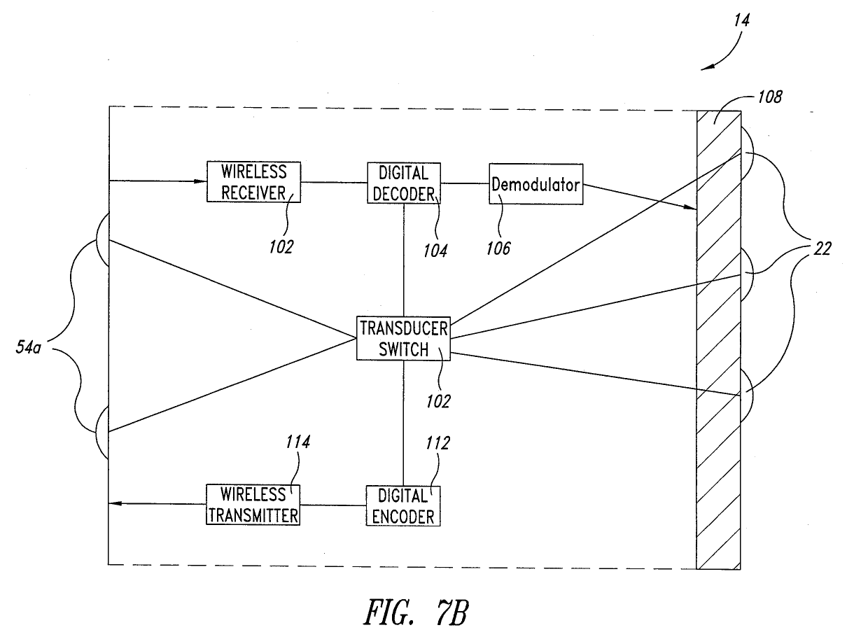

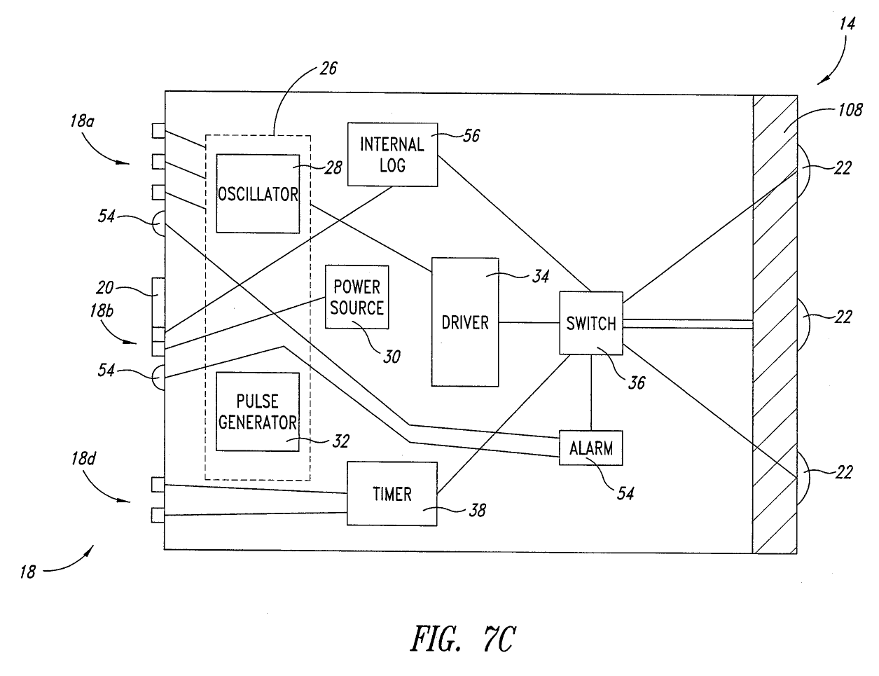

Referring to FIGS. 7A, 7B, and 7C, the transducer 14a may be wirelessly coupled to a control module 18 that communicates with the transducer 14a via wireless communication. Examples of wireless communication including for example, optical connections, ultraviolet connections, infrared, BLUETOOTH®, Internet connections, network connections, and the like. In some embodiments, the device 12 comprises a control module and at least one wireless transducer or transducer array. Internal batteries may render the device 12 cordless and allow for maximum portability of the power the control module 18 and the transducer 14. Alternatively, the control module may remain stationary in a fixed location such that the control module 18 can be powered by coupling it to an electrical socket.

As shown in FIGS. 7A and 7B, the waveform generator 26 communicates with a first digital encoder 90 which converts the signal received from the waveform generator 26 to a digital signal and delivers the digitally encoded signal to a first wireless transmitter 92. The first wireless transmitter 92 in the control module is in wireless communication with a first wireless receiver 102 in the transducer 14a. Any construction of the control module and wireless transducer 14a suitable for effecting wireless communication can be used in this embodiment.

One or more input device elements of control module 18 may communicate with a waveform generator 26. In some embodiments, the waveform generator 26 may also include an amplitude modulator 94 for modifying the waveform into a form transmittable by, for example, radio waves, as is generally known. The waveform generator 26 can include a single variable frequency RF oscillator programmed to deliver different frequencies based on the type of tissue selected, or a plurality of RF oscillators, one for each type of tissue.

In one exemplary embodiment, a first wireless transmitter 92 can deliver a single wireless signal receivable by each of the first wireless receivers 102 in the one or more transducers 14a, such that each transducer 14a outputs the same waveform. Alternatively, the first wireless transmitter 92 can deliver a plurality of wireless signals, and the first wireless receivers 102 can be configured to receive a single signal unique to each individual transducer 14a such that each transducer 14a or array will output a distinct waveform. In some embodiments, the control module can include at least one input device element for inputting one or more treatment parameters associated with each of the transducer 14a. Such an embodiment, allows the physician or user to control a plurality of transducers 14a or transducer arrays from a remote location, and enables treatment of more than one patient at a time.

As shown in FIG. 7B, each transducer 14a and/or array may comprise a first wireless receiver 102 for receiving wireless signals from the first wireless transmitter 92 in the control module. The first wireless receiver 102 of transducer 14a delivers the signal to a first digital decoder 104, which decodes the signal and delivers the resulting analog information to a demodulator 106. The demodulator 106 demodulates the analog and/or information and delivers the waveform to the treatment surface 108 of the transducer 14a.

Each transducer 14a may also comprise at least one sensor 22 for sensing the temperature of the patient's skin, target tissue within the body, and transducer treatment element during treatment or for sensing a loss of contact between the transducer and the patient's skin. The sensor 22 communicates with a transducer switch 110, and the transducer switch 110 is configured to cease delivery of waveform information from the first digital decoder 104 to the treatment surface. The transducer may further include trouble shouting guides, alarms, indicators, and the like for alerting the user to a malfunction. For example, the transducer may include at least one light emitting diode (LED) on the visible surface of the transducer that lights up or flashes when a malfunction occurs. Alternatively, the transducer may include an audible alarm that alerts the user to the malfunction. The transducer may also include both an audible and visible alarm, such as a flashing LED coupled with an audible alarm.

The transducer switch 110 may also communicates with a second digital encoder 112. The transducer switch 110 receives information regarding the transducer 14 use and malfunctions and delivers the information to the second digital encoder 112. The second digital encoder 112 then digitally encodes the information and delivers it to a second wireless transmitter 114 in the transducer 14a, which delivers the digitally encoded signal to a second wireless receiver 116 in the delivery device 12.

In some embodiments, a self-contained transducer device 12 adapted to generate and transmit a waveform for a selected tissue type may include a control module and a transducer. The self-contained transducer device 12 can be powered by an internal battery pack, rendering the self-contained transducer device 12 cordless, as well as maximizing device 12 portability. In some embodiments, the self-contained transducer device 12 may be designed to treat a single tissue type at a given time. The transducer device 12 may include a first input device element for selecting tissue type, a second input device elements for selecting injury type, and a third input device elements for setting the treatment time. The self-contained transducer device 12 may be operable to vary the waveform based on the type of tissue and the type of injury selected.

In some embodiments, each of the first input device elements, second input device elements, and third input device elements communicate with a waveform generator 26. In one embodiment, only one RF oscillator designed to deliver the appropriate frequency is provided. In another embodiment, the waveform generator 26 includes a plurality of RF oscillators 28, one for each tissue type. However, because the self-contained transducer device 12 is designed for maximum portability and to treat a single tissue type at a time, embodiments with only one RF oscillator 28 may be more desirable.

In some embodiments, the device 12 may include a single oscillator 28 in the form of an RF oscillator. The RF oscillator may be programmed to deliver a frequency based on the tissue type selected with the first input device element. Alternatively, the RF oscillator 28 may be pre-programmed to deliver a single, specified frequency that is not user-selectable. In this embodiment, the need for the first input device element is eliminated.

In some embodiments, device 12 is operable to provide therapeutic treatment of only a single tissue type. In some embodiments, a kit comprising at least two devices 12, each designed to treat a different tissue type, may be provided. For example, one kit may include at least one device 12 designed to treat bone, and at least one device 12 designed to treat tendons or ligaments. Alternatively, a kit may include a plurality of devices 12 including at least one device for treating each type of tissue.

In some embodiments, more than one waveform may be used in each treatment session. If more than one waveform is used, the user may select how the device moves from one waveform to the next. Each waveform may be a distinct treatment point used for a selected period of time. Alternatively, the waveforms may be points on a signal wave (e.g., a sine wave, and the like) and the device may gradually sweep from one waveform to the next in discrete increments. For example, the device will gradually sweep from one waveform to the next in no less than about 5 and no more than about 10 steps. In some embodiments, the treatment time is divided generally equally between each step in the transition between waveforms. During the transition from one waveform to the next, only the pulse intensity will change until the next selected pulse intensity is achieved, at which time the remaining characteristics of the next waveform will be generated. This process continues until the treatment elapses.

The variant waveforms of the pulses and pulse series generated by the device 12 enable a generally static treatment of injuries. Previously, treatment with ultrasound required rapid and substantial movement of the treatment elements (e.g., transducers) in order to avoid thermal damage to the target and surrounding tissue. The need for constantly moving the treatment elements to maintaining the thermal energy at or bellow a desire amount is reduced or substantially eliminated, in part, by varying the waveform and providing temperature sensors 22 to monitor tissue temperature during treatment. This generally static treatment of injuries significantly increases the ease of use of the ultrasound devices 12, and may significantly reduce user error, as well a thermal tissue damage.

During each therapeutic treatment session, the ultrasonic treatment waveform may include multiple waveform segments, each segment having different characteristics. The total treatment time is divided between these segments as determined by the user. The user may alter the waveform or the treatment time of each segment with the first, second and third input device elements described above. Alternatively, the waveform segments can be pre-programmed into the device by the manufacturer and the user can select the pre-programmed waveform.

In some embodiments, the region of the body of the subject to be treated is first shaved and a coupling gel is applied to the skin. The transducer 14 is then placed over the coupling gel and the device 12 is used to transmit ultrasound energy to the treatment area. If the treatment area is located underneath a cast or the like, a window is cut in the cast and the coupling gel and transducer are applied to the area through the window. The first and second input device elements 12 and 14 are used to select tissue type and injury type, and treatment is commenced. During treatment, the transducer 14 is held in place by techniques well know in the relevant art.

Many injuries have both acute and chronic components. As a result, treatment of such injuries may begin by treating the injured tissue for the acute component, and later treating the tissue for the chronic component. Alternatively, the tissue may be treated for the chronic injury first and the acute injury later. In another embodiment, both the acute and the chronic components of the injury are treated by varying the intensity, frequency, pulse intensity, duration, ratio, and repetition rate during each treatment session.

Tables 1 and 2 below list exemplary treatment parameters for each tissue type. The parameters listed in Tables 1 and 2 are for the treatment of acute injuries to the indicated tissue type with uni-variant waveform segments through which the treatment device cycles throughout the treatment time. Increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type. While a single, fixed frequency ultrasonic output wave may be used to therapeutically treat any number of tissue types, varying the frequency based upon the depth of the target tissue within the body may, for example, more accurately focus and concentrate the deposition of the ultrasound energy on the desired target tissue. This adjustment of the ultrasonic output wave's frequency based upon target tissue depth within the body may render the therapeutic ultrasound treatment more effective.

TABLE 1
Treatment parameters for acute injuries - Uni-variant waveform segments

| | | TENDON/ | | |
|---|---|---|---|---|
| | BONE | LIGAMENT | JOINT | MUSCLE |
| FREQUENCY | 1.5 MHz | 3 MHz | 2.5 MHz | 1 MHz |
| PULSE RATIO | 1:4 | 1:4 | 1:4 | 1:4 |
| PULSE FREQUENCY | 1,000 Hz | 1,000 Hz | 1,000 Hz | 1,000 Hz |
| TOTAL | 30 min. | 35 min. | 35 min. | 30 min. |
| TREATMENT TIME | | | | |
| PULSE DURATION | 200 μsec | 200 μsec | 200 μsec | 200 μsec |

TABLE 2
Pulse intensities for acute injuries - Uni-variant waveform segments

| | | TENDON/ | | |
|---|---|---|---|---|
| | BONE | LIGAMENT | JOINT | MUSCLE |
| PHASE 1 | begin at | begin at | begin at | begin at |
| | 30 mW/cm², | 30 mW/cm², | 50 mW/cm², | 100 mW/cm², |
| | increase to | increase to | increase to | increase to |
| | 50 mW/cm² and | 50 mW/cm² and | 300 mW/cm² and | 500 mW/cm² and |
| | decrease back to | decrease back to | decrease back to | decrease back to |
| | 30 mW/cm² over 5 min. | 30 mW/cm² over 5 min. | 50 mW/cm² over 5 min. | 100 mW/cm² over |
| | period | period | period | 5 min. period |
| PHASE 2 | begin at | begin at | begin at | begin at |
| | 30 mW/cm², | 50 mW/cm², | 30 mW/cm², | 30 mW/cm², |
| | increase to | increase to | increase to | increase to |
| | 100 mW/cm² and | 300 mW/cm² and | 100 mW/cm² and | 200 mW/cm² and |
| | decrease back to | decrease back to | decrease back to | decrease back to |
| | 30 mW/cm² over 5 min. | 50 mW/cm² over 5 min. | 30 mW/cm² over 5 min. | 30 mW/cm² over |
| | period | period | period | 5 min. period |
| PHASE 3 | begin at | begin at | begin at | begin at |
| | 30 mW/cm², | 30 mW/cm², | 50 mW/cm², | 50 mW/cm², |
| | increase to | increase to | increase to | increase to |
| | 50 mW/cm² and | 100 mW/cm² and | 200 mW/cm² and | 300 mW/cm² and |
| | decrease back to | decrease back to | decrease back to | decrease back to |
| | 30 mW/cm² over 5 min. | 30 mW/cm² over 5 min. | 50 mW/cm² over 5 min. | 50 mW/cm² over |
| | period | period | period | 5 min. period |
| PHASE 4 | begin at | begin at | begin at | begin at |
| | 30 mW/cm², | 50 mW/cm², | 30 mW/cm², | 100 mW/cm², |
| | increase to | increase to | increase to | increase to |
| | 100 mW/cm² and | 300 mW/cm² and | 100 mW/cm² and | 500 mW/cm² and |
| | decrease back to | decrease back to | decrease back to | decrease back to |
| | 30 mW/cm² over 5 min. | 50 mW/cm² over 5 min. | 30 mW/cm² over 5 min. | 100 mW/cm² over |
| | period | period | period | 5 min. period |
| PHASE 5 | begin at | begin at | begin at | begin at |
| | 30 mW/cm², | 30 mW/cm², | 50 mW/cm², | 30 mW/cm², |
| | increase to | increase to | increase to | increase to |
| | 50 mW/cm² and | 50 mW/cm² and | 300 mW/cm² and | 200 mW/cm² and |
| | decrease back to | decrease back to | decrease back to | decrease back to |
| | 30 mW/cm² over 5 min. | 30 mW/cm² over 5 min. | 50 mW/cm² over 5 min. | 30 mW/cm² over |
| | period | period | period | 5 min. period |
| PHASE 6 | begin at | begin at | begin at | begin at |
| | 30 mW/cm², | 50 mW/cm², | 30 mW/cm², | 50 mW/cm², |
| | increase to | increase to | increase to | increase to |
| | 100 mW/cm² and | 300 mW/cm² and | 100 mW/cm² and | 300 mW/cm² and |
| | decrease back to | decrease back to | decrease back to | decrease back to |
| | 30 mW/cm² over 5 min. | 50 mW/cm² over 5 min. | 30 mW/cm² over 5 min. | 50 mW/cm² over |
| | period | period | period | 5 min. period |
| PHASE 7 | — | begin at | begin at | — |
| | | 30 mW/cm², | 50 mW/cm², | |
| | | increase to | increase to | |
| | | 100 mW/cm² and | 200 mW/cm² and | |
| | | decrease back to | decrease back to | |
| | | 30 mW/cm² over 5 min. | 50 mW/cm² over 5 min. | |
| | | period | period | |
| TOTAL | 30 min. | 35 min. | 35 min. | 30 min. |
| TREATMENT | | | | |
| TIME | | | | |

In the non-limiting examples of uni-variant waveforms listed in Tables 1 and 2, the pulse intensity is variable and begins at an initial setting (e.g., 30 mW/cm²) and increases to a peak intensity (e.g., 50 mW/cm²) in increments of no less than about 5 and no more than about 10 intensity points per treatment interval. Furthermore, as indicated by Table 2, treatment of injuries occurs in phases that may have different initial and peak intensities. As shown in Table 2, each phase is about 5 minutes in length, in which time the intensity increases from the initial intensity to the peak intensity and decreases back to the initial intensity. The treatment time may vary depending on the type of tissue being treated. In addition, although not exemplified in Tables 1 and 2, the treatment time may vary depending on the type of injury sustained by the indicated tissue as well as the chronicity of the injury being treated. It is understood that all parameters of the waveform may be user-selectable and variable. These variable waveform characteristics include intensity, frequency, pulse frequency, pulse intensity, pulse duration, pulse ratio, and pulse repetition rate. This variability in waveform characteristics enables the generally static application of ultrasonic therapy.

Tables 3 through 6 below list other exemplary treatment parameters for each tissue type. Table 3 lists exemplary parameters for the treatment of bone with multi-variant waveform segments for a total treatment time of 45 minutes. Table 4 lists exemplary parameters for the treatment of tendons and ligaments with multi-variant waveform segments for a total treatment time of 40 minutes. Table 5 lists exemplary parameter for the treatment of joints with multi-variant waveform segments for a total treatment time of 40 minutes. Table 6 lists exemplary parameters for the treatment of muscle with multi-variant waveform segments for a total treatment time of 40 minutes. Like those of Tables 1 and 2, the parameters listed in Tables 3 through 6 are for the treatment of acute injuries to the indicated tissue type with multi-variant waveform segments through which the treatment device cycles throughout the treatment time. As with the uni-variant waveforms listed in Tables 1 and 2, increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type.

TABLE 3
Treatment parameters for acute injuries to bone -
Multi-variant waveform segments

| | PHASE 1 | PHASE 2 | PHASE 3 | PHASE 4 | PHASE 5 | PHASE 6 |
|---|---|---|---|---|---|---|
| FREQUENCY | 1.5 MHz | 1.5 MHz | 1.5 MHz | 1.5 MHz | 1.5 MHz | 1.5 MHz |
| PULSE | 1:4 | 1:2 | 1:3 | 1:4 | 1:1 | 1:4 |
| RATIO | | | | | | |
| PULSE | 1,000 Hz | 835 Hz | 2,500 Hz | 1,000 Hz | 5,000 Hz | 1,000 Hz |
| FREQUENCY | | | | | | |
| PULSE | 200 μsec | 400 μsec | 100 μsec | 200 μsec | 100 μsec | 200 μsec |
| DURATION | | | | | | |
| PULSE | begin at 30 mW/cm² | begin at | begin at | begin at 30 mW/cm² | begin at 30 mW/cm² | begin at |
| INTENSITY | and | 50 mW/cm² | 30 mW/cm² | and | and | 30 mW/cm² |
| | increase to | and | and | increase to | increase to | and |
| | 50 mW/cm² | increase | increase | 80 mW/cm² | 100 mW/cm² | increase |
| | and | to 100 mW/cm² | to 200 mW/cm² | and | and | to 50 mW/cm² |
| | decrease | and | and | decrease | decrease | and |
| | back to 30 mW/cm² | decrease | decrease | back to 30 mW/cm² | back to 30 mW/cm² | decrease |
| | | back to | back to 30 mW/cm² | | | back to |
| | | 50 mW/cm² | | | | 30 mW/cm² |
| SEGMENT | 10 min | 5 min | 5 min | 10 min | 5 min | 10 min |
| TREATMENT | | | | | | |
| TIME | | | | | | |

TABLE 4
Treatment parameters for acute injuries to tendons and ligaments -
Multi-variant waveform segments

| | PHASE 1 | PHASE 2 | PHASE 3 | PHASE 4 | PHASE 5 | PHASE 6 |
|---|---|---|---|---|---|---|
| FREQUENCY | 3 MHz | 3 MHz | 3 MHz | 3 MHz | 3 MHz | 3 MHz |
| PULSE | 1:4 | 1:2 | 1:4 | 1:3 | 1:4 | 1:1 |
| RATIO | | | | | | |
| PULSE | 1,000 Hz | 835 Hz | 1,000 Hz | 2,500 Hz | 1,000 Hz | 5,000 Hz |
| FREQUENCY | | | | | | |
| PULSE | 200 μsec | 400 μsec | 200 μsec | 100 μsec | 200 μsec | 100 μsec |
| DURATION | | | | | | |
| PULSE | begin at | begin at | begin at | begin at | begin at | begin at 30 mW/cm² |
| INTENSITY | 30 mW/cm² | 50 mW/cm² | 30 mW/cm² | 30 mW/cm² | 30 mW/cm² | and |
| | and | and | and | and | and | increase to |
| | increase to | increase to | increase | increase | increase | 100 mW/cm² |
| | 50 mW/cm² | 300 mW/cm² | to 80 mW/cm² | to 200 mW/cm² | to 50 | and |
| | and | and | and | and | mW/cm² | decrease |
| | decrease | decrease | decrease | decrease | and | back to 30 mW/cm² |
| | back to 30 mW/cm² | back to 50 mW/cm² | back to 30 mW/cm² | back to 30 mW/cm² | decrease | |
| | | | | | back to | |
| | | | | | 30 mW/cm² | |
| SEGMENT | 10 min | 5 min | 5 min | 5 min | 10 min | 5 min |
| TREATMENT | | | | | | |
| TIME | | | | | | |

TABLE 5
Treatment parameters for acute injuries to joints -
Multi-variant waveform segments

| | PHASE 1 | PHASE 2 | PHASE 3 | PHASE 4 | PHASE 5 | PHASE 6 |
|---|---|---|---|---|---|---|
| FREQUENCY | 2.5 MHz | 2.5 MHz | 2.5 MHz | 2.5 MHz | 2.5 MHz | 2.5 MHz |
| PULSE | 1:4 | 1:4 | 1:2 | 1:3 | 1:4 | 1:1 |
| RATIO | | | | | | |
| PULSE | 2,000 Hz | 1,000 Hz | 835 Hz | 1,250 Hz | 1,000 Hz | 5,000 Hz |
| FREQUENCY | | | | | | |
| PULSE | 100 μsec | 200 μsec | 400 μsec | 200 μsec | 200 μsec | 100 μsec |
| DURATION | | | | | | |
| PULSE | begin at | begin at | begin at 50 mW/cm² | begin at 30 mW/cm² | begin at | begin at 50 mW/cm² |
| INTENSITY | 50 mW/cm² | 30 mW/cm² | and | and | 30 mW/cm² | and |
| | and | and | increase to | increase to | and | increase to |
| | increase | increase | 200 mW/cm² | 100 mW/cm² | increase | 100 mW/cm² |
| | to 300 mW/cm² | to 80 mW/cm² | and | and | to 50 mW/cm² | and |
| | | and | decrease | decrease | | decrease |
| | | decrease | back to 50 mW/cm² | back to 30 mW/cm² | | back to 30 mW/cm² |
| | | back to 30 mW/cm² | | | | |
| SEGMENT | 5 min | 10 min | 5 min | 5 min | 10 min | 5 min |
| TREATMENT | | | | | | |
| TIME | | | | | | |

TABLE 6
Treatment parameters for acute injuries to muscle -
Multi-variant waveform segments

| | PHASE 1 | PHASE 2 | PHASE 3 | PHASE 4 | PHASE 5 | PHASE 6 |
|---|---|---|---|---|---|---|
| FREQUENCY | 1 MHz | 1 MHz | 1 MHz | 1 MHz | 1 MHz | 1 MHz |
| PULSE | 1:1 | 1:4 | 1:2 | 1:4 | 1:1 | 1:4 |
| RATIO | | | | | | |
| PULSE | 1,000 Hz | 1,000 Hz | 835 Hz | 2,000 Hz | 1,665 Hz | 1,000 Hz |
| FREQUENCY | | | | | | |
| PULSE | 500 μsec | 200 μsec | 400 μsec | 100 μsec | 300 μsec | 200 μsec |
| DURATION | | | | | | |
| PULSE | begin at | begin at 30 mW/cm² | begin at | begin at 30 mW/cm² | begin at | begin at |
| INTENSITY | 500 mW/cm² | and | 300 mW/cm² | and | 400 mW/cm² | 30 mW/cm² |
| | and | increase to | and | increase to | and | and |
| | increase | 100 mW/cm² | increase to | 200 mW/cm² | increase to | increase |
| | to 1.5 W/cm² | and | 1 W/cm² | and | 1.2 W/cm² | to 80 mW/cm² |
| | | decrease | | decrease | and decrease | and |
| | | back to 30 mW/cm² | | back to 30 mW/cm² | back to 400 mW/cm² | decrease |
| | | | | | | back to |
| | | | | | | 30 mW/cm² |
| SEGMENT | 3 min | 10 min | 5 min | 7 min | 5 min | 10 min |
| TREATMENT | | | | | | |
| TIME | | | | | | |

Although principally described for treating injuries to certain tissues, device 12 can be used for any suitable purpose. For example, the devices can be used to treat joints and joint capsules. In addition, the devices can be used to promote the differentiation and/or maturation of stem cells in culture or within a human or animal body.

In some embodiments, stem cell therapy is used in conjunction with therapeutic ultrasound treatment. Typically, the stem cells are first harvested from bone marrow, adipose tissue, peripheral blood from an embryo and/or an umbilical cord, or other tissue. The harvested stem cells are then implanted and/or transplanted into the injured target tissue by injecting them intralesionally, intravenously, intrathecally, intraarticularly, and the like. After injection of the stem cells, the treatment area is treated with ultrasound therapy. Upon treatment of the injected tissue with ultrasound therapy, the injured tissue, the stem cells, and the ultrasound synergistically may promote tissue healing, growth, regeneration, and repair.

According to this exemplary embodiment, the stem cells can be injected into the tissue before, after or concurrently with the ultrasound treatment, and the ultrasound treatment would utilize pulsed or continuous waveforms having frequencies ranging from about 50 kHz to about 3 MHz and intensities ranging from about 20 mW to about 3 W. These methods of treatment (e.g., stem cell therapy used in conjunction with ultrasound treatment) can be used to treat several tissue types, including but not limited to bone, muscle, tendons, ligaments, and cartilage.

In some embodiments, a bioactive agents is used in conjunction with ultrasound therapy and stem cell therapy. The term “bioactive agent” generally refers to one or more compounds, molecules, or treatments that elicit a biological response from any host, animal, vertebrate, or invertebrate, including for example fish, mammals, amphibians, reptiles, birds, and humans. Examples of bioactive agents include therapeutic agents, pharmaceutical agents, pharmaceuticals (e.g., a drug, a therapeutic compound, pharmaceutical salts, and the like) non-pharmaceuticals (e.g., cosmetic substance, and the like), any of the growth factor families (e.g., insulin-like growth factors, tissue growth factors, bone growth factors, and the like), a local or general anesthetic or painkiller, an antigen or a protein or peptide such as insulin, a chemotherapy agent, an anti-tumor agent, combinations thereof, and the like

The term “active agent” further refers to the active agent, as well as its pharmacologically active salts, pharmaceutically acceptable salts, prodrugs, metabolites, analogs, and the like. Non-limiting examples of suitable one or more bioactive agents include dexamethasone, TGF-beta, IGF-1, BMP-2, CDMP-2, FGF-1, all members of the bone morphogenic protein family including all cartilage-derived morphogenic proteins, all members of the tissue and transforming growth factor families, all member of the insulin-like growth factor family, all members of the fibroblast growth factor family, hyaluronans and their derivatives, and any other growth factors appropriate for assisting the differentiation and/or maturation of stem cells.

In some embodiments, at least one of the stem cells, target tissue, and body surrounding the target tissue is treated with a one or more bioactive agents. The bioactive agent assist the differentiation and/or maturation of the stem cells, and are added to the stem cells, tissue, or body either before and/or after the stem cells are injected in the target tissue and before, after or during the ultrasound therapy. Non-limiting examples of suitable one or more bioactive agents include dexamethasone, TGF-beta, IGF-1, BMP-2, CDMP-2, FGF-1, all members of the bone morphogenic protein family including all cartilage-derived morphogenic proteins, all members of the tissue and transforming growth factor families, all member of the insulin-like growth factor family, all members of the fibroblast growth factor family, hyaluronans and their derivatives, and any other growth factors appropriate for assisting the differentiation and/or maturation of stem cells.

One exemplary method for treating injured tissue includes first harvesting stem cells from bone marrow, adipose tissue, peripheral blood from an embryo, fetus, adult or an umbilical cord, or other tissue. The stem cells are treated with the one or more bioactive agents either in vitro or in vivo. The treatment area within the body may also be treated with the one or more bioactive agents before and/or after stem cell injection, and before, after or concurrently with the ultrasound treatment. The stem cells are injected into the treatment area either before and/or after being treated with the one or more bioactive agents. As noted above, the stem cells may be injected in any suitable manner, such as intralesionally, intravenously, intramuscularly, intrathecally, intraarticularly, and the like. The treatment area is then treated with ultrasound. Upon treatment of the injected tissue with ultrasound therapy, the injured tissue, the stem cells, the one or more bioactive agents and the ultrasound synergistically promote tissue healing, growth, regeneration, and repair. These methods of treatment (e.g., stem cell therapy used in conjunction with ultrasound treatment) can be used to treat several tissue types, including but not limited to bone, muscle, tendons and/or ligaments and cartilage.

FIG. 9 shows an exemplary method 200 of treating at least one condition associated with injured tissue in a subject.

At 202, the method includes contacting a location on a biological interface of the subject with an ultrasound delivery device 12. One or more ultrasound transducers 14 are operable for providing an ultrasonic signal comprising at least a first waveform segment and a second waveform segment, the first waveform segment having at least one of an intensity, frequency, pulse intensity, pulse duration, pulse ratio, or pulse repetition rate different from the second segment.

At 204, the method further includes applying a sufficient amount of current to emit a therapeutically effective amount of ultrasonic energy from the ultrasound delivery device 12.

In some embodiments, applying a sufficient amount of current comprises applying enough current to emit a therapeutically effective amount of ultrasonic energy for at least one interval in a 24-hour period, the interval ranging from about 5 minutes to about 60 minutes. In some other embodiments, applying a sufficient amount of current comprises applying a sufficient amount current to emit a therapeutically effective amount of ultrasonic energy for at least one to three intervals in a 24-hour period, each interval independently ranging from about 5 minutes to about 60 minutes.

In some embodiments, each interval independently ranges from about 20 minutes to about 45 minutes.

At 206, the method further includes providing at least one control parameter selected from a tissue type, a treatment area, a lesion depth, a degree of injury, a treatment type, a duration type, and one or more waveform characteristics.

In some embodiments, the treatment type is selected from continuous or pulsed, and the one or more waveform characteristics are selected from an intensity, frequency, pulse intensity, pulse duration, pulse ratio, and pulse repetition rate. In some embodiments, the degree of injury is selected from an acute injury, a sub-acute injury, and a chronic injury. In some embodiments, the tissue type is selected from bone, cartilage, muscle, tendon, ligament, and stem cells, or combinations thereof.

At 208, the method further includes providing stem cell therapy before, during, and/or after emitting the therapeutically effective amount of ultrasonic energy. In some embodiments, providing stem cell therapy includes providing stem cell implantation, stem cell transplantation, stem cell delivery, and the like to the injured tissue, or combinations thereof. In some embodiments, providing stem cell therapy further includes administering one or more bioactive agents to the injured tissue.

The one or more bioactive agents may be selected from TGF-beta, IGF-1, BMP-2, CDMP-2, FGF-1, bone morphogenic proteins, cartilage-derived morphogenic proteins, tissue growth factors, transforming growth factors, insulin-like growth factors, fibroblast growth factors and hyaluronans, or combinations thereof.

At 210, the method further includes treating the injured tissue with one or more bioactive agents before, during, and/or after providing stem cell therapy to the injured tissue, and treating the injured tissue with one or more bioactive agents before, during, and/or after emitting the therapeutically effective amount of ultrasonic energy.

As one skill in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the waveform generators, treatment control modules, RF generators, and structures described above while employing additional ones of the waveform generators, treatment control modules, RF generators, and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. An ultrasound therapy device for delivering ultrasonic treatment to a biological entity, comprising:
a waveform generator configured to generate a first driving signal, the first driving signal comprising at least a first waveform segment and a second waveform segment different from the first waveform segment;
one or more transducers electrically coupled to the waveform generator, the one or more transducers configured to receive the first driving signal and to generate a first ultrasonic signal based in part on the first driving signal;
one or more sensors operable to determine at least one physiological characteristic of the biological entity; and
a controller electrically coupled the waveform generator and the one or more sensors, the controller configured to perform a comparison of the determined at least one physiological characteristic of the biological entity to stored reference data, and to generate a response based in part on the comparison.

2. The device of claim 1, wherein the first waveform segment has at least one of an intensity, a frequency, a pulse intensity, a pulse duration, a pulse frequency, a pulse ratio, or a pulse repetition rate different from the second waveform segment.

3. The device of claim 1, wherein at least one of the first waveform or the second waveform segments comprises a continuous waveform.

4. The device of claim 1, wherein the first and the second waveform segments are each independently selected from one or more single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, or combinations thereof.

5. The device of claim 1, wherein the first driving signal comprises a repeating signal comprising one or more waveforms selected from single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, single-frequency formed waves, multi- frequency formed waves, or combinations thereof.

6. The device of claim 1, wherein the generated first ultrasonic signal compromise at least one of a continuous or a pulsed ultrasonic treatment wave, or combinations thereof.

7. The device of claim 1, wherein an intensity of the first ultrasonic signal ranges from about 0.02 W/cm² to about 3 W/cm².

8. The device of claim 1, wherein an intensity of the first ultrasonic signal ranges from about 0.02 W/cm² to about 1.5 W/cm².

9. The device of claim 1, wherein an intensity of the first ultrasonic signal ranges from about 0.4 W/cm² to about 1.5 W/cm².

10. The device of claim 1, wherein a pulse ratio of the first ultrasonic signal ranges from about to about 1:1 to about 1:20.

11. The device of claim 1, wherein a frequency of the first ultrasonic signal ranges from about 0.05 MKHz to about 3 MHz.

12. The device of claim 1, wherein a pulse repetition rate of the first ultrasonic signal ranges from about 50 KHz to about 10,000 Hz.

13. The device of claim 1, wherein a pulse duration of the first ultrasonic signal ranges from about 10 μs to about 2,500 μs.

14. The device of claim 1 wherein the first ultrasonic signal comprises a pulse duration ranging from about 10 μs to about 2,500 μs, a pulse ratio ranging from about 1:1 to about 1:8, a pulse repetition rate ranging from about 50 Hz to about 10,000 Hz, and a pulse intensity ranging about 0.02 W/cm² to about 3 W/cm².

15. The device of claim 1, wherein the first ultrasonic signal comprises a pulse repetition rate ranging from about 500 Hz to about 2,500 Hz, and a pulse duration ranging from about 100 μs to about 500 μs.

16. The device of claim 1, wherein the waveform generator is further configured to cycle through the at least first and the second waveform segments for a limited treatment time.

17. The device of claim 1, wherein at least one of the one or more sensors is operable to generate a response based in part on whether the one or more transducers are physically contacting a treatment site on the biological subject.

18. The device of claim 1, wherein the one or more transducers take the form of a transducer array.

19. The device of claim 1, wherein the waveform generator is configured to generate the first driving signal based in part on a user input.

20. The device of claim 19, wherein the user input comprises at least one of a continuous output, a pulsed output, an intensity, a frequency, a pulse ratio, a pulse intensity, a pulse duration, a pulse frequency, a pulse repetition rate, a treatment type, a treatment time, a treatment time increase or decrease, a treatment interval rate, a lesion depth, a degree of tissue injury, and a tissue type, or combinations thereof.

21. The device of claim 20, wherein the degree of tissue injury is selected from an acute injury, a sub-acute injury, a chronic injury; and the tissue type is selected from bone, cartilage, muscle, tendon, ligament, and stem cells, or combinations thereof.

22. The device of claim 1, wherein the generated response includes at least one of a response signal, a comparison plot, a treatment code, a diagnostic code, a test code, an alarm, a change to a treatment parameter, and a response signal operable to terminate the first driving signal.

23. The device of claim 1, wherein the controller is further configured to track at least one treatment parameter selected from a treatment type, a treatment time, a treatment duration, a lesion depth, a degree of tissue injury, a tissue type, an intensity, a frequency, a pulse frequency, a pulse ratio, a pulse intensity, a pulse duration, a pulse frequency, a pulse repetition rate, a continuous waveform frequency, and a continuous waveform intensity.

24. The device of claim 1, wherein the least one physiological characteristic is selected from an impedance, a temperature, a density, and/or a fat content of the biological entity.

25. The device of claim 1, wherein the stored reference data comprises at least one of characteristic physiological data, characteristic impedance data, characteristic temperature data, characteristic density data, and characteristic fat content data.

26. An ultrasound therapy system for delivering ultrasonic treatment to a biological entity, comprising:
an inductive power supply including a primary winding operable to produce a varying magnetic field; and
an ultrasound delivery device, the ultrasound delivery device comprising a waveform generator configured to generate a first driving signal, the first driving signal comprising at least a first waveform segment and a second waveform segment, the second waveform segment different from the first; a secondary winding electrically coupled to the waveform generator and operable for providing a potential to the waveform generator in response to a varying electromagnetic field applied to the secondary winding; and one or more transducers electrically coupled to the waveform generator, the one or more transducers configured to receive the first driving signal and to generate a first ultrasonic signal based in part on the first driving signal.

27. The system of claim 26, wherein the ultrasound delivery device is physically distinct from the inductive power supply.

28. The system of claim 26, wherein the inductive power supply is operable to provide at least one of an alternating current or a pulsed direct current to the primary winding.

29. The system of claim 26, wherein the ultrasound therapy device includes a rechargeable power source electrically coupled to the waveform generator, and electrically coupled in parallel with the secondary winding to receive a charge thereby.

30. The system of claim 26, wherein the rechargeable power source sinks and sources voltage to maintain a steady state operation of the ultrasound therapy device.

31. The ultrasound therapy device of claim 30, the rechargeable power source comprises at least one of a button cell, a chemical battery cell, a fuel cell, a secondary cell, a lithium ion cell, a nickel metal hydride cell, a super-capacitor, a thin film secondary cell, an ultra-capacitor, and a zinc air cell.

32. The system of claim 26, wherein the inductive power supply is operable to manage a duty cycle associated with emitting a therapeutically effective amount of ultrasonic energy from the ultrasound delivery device.

33. The ultrasound therapy device of claim 26, further comprising:
one or more sensors operable to determine at least one physiological characteristic of the biological entity; and
a controller electrically coupled the waveform generator and the one or more sensors, the controller configured to perform a comparison of the determined at least one physiological characteristic of the biological entity to stored reference data, and to generate a response based in part on the comparison.

34. A method of treating at least one condition associated with injured tissue in a subject, comprising:
contacting a location on a biological interface of the subject with an ultrasound delivery device comprising one or more ultrasound transducers, the at least the one or more ultrasound transducers operable for providing an ultrasonic signal comprising at least a first waveform segment and a second waveform segment; the first waveform segment having at least one of an intensity, a frequency, a pulse intensity, a pulse duration, a pulse ratio, or a pulse repetition rate different from the second segment; and
applying a sufficient amount of current to emit a therapeutically effective amount of ultrasonic energy from the ultrasound delivery device.

35. The method of claim 34, wherein applying a sufficient amount of current comprises applying a sufficient amount of current to emit a therapeutically effective amount of ultrasonic energy for at least one interval in a 24 hour period, the interval ranging from about 5 minutes to about 60 minutes.

36. The method of claim 34, wherein applying a sufficient amount of current comprises applying a sufficient amount current to emit a therapeutically effective amount of ultrasonic energy for at least one to three intervals in a 24 hour period, each interval independently ranging from about 5 minutes to about 60 minutes.

37. The method of claim 36, wherein each interval independently ranges from about 20 minutes to about 45 minutes.

38. The method of claim 34, further comprising:
providing at least one control parameter, the at least one control parameter selected from a tissue type, a treatment area, a lesion depth, a degree of injury, a treatment type, a duration type, and one or more waveform characteristics.

39. The method of claim 38, wherein the treatment type is selected from continuous or pulsed, and the one or more waveform characteristics are selected from an intensity, a frequency, a pulse intensity, a pulse duration, a pulse ratio, and a pulse repetition rate.

40. The method of claim 38, wherein the degree of injury is selected from an acute injury, a sub-acute injury, and a chronic injury.

41. The method of claim 38, wherein the tissue type is selected from bone, cartilage, muscle, tendon, ligament, and stem cells, or combinations thereof.

42. The method of claim 34, further comprising:
providing stem cell therapy before, during, and/or after emitting the therapeutically effective amount of ultrasonic energy.

43. The method of claim 42, wherein providing stem cell therapy includes providing stem cell implantation, stem cell transplantation, or stem cell delivery to the injured tissue, or combinations thereof.

44. The method of claim 43, wherein providing stem cell therapy further includes administering one or more bioactive agents to the injured tissue.

45. The method of claim 44, wherein the one or more bioactive agents are selected from TGF-beta, IGF-1, BMP-2, CDMP-2, FGF-1, bone morphogenic proteins, cartilage-derived morphogenic proteins, tissue growth factors, transforming growth factors, insulin-like growth factors, fibroblast growth factors and hyaluronans, or combinations thereof.

46. The method of claim 34, further comprising:
treating the injured tissue with one or more bioactive agents before, during, and/or after providing stem cell therapy to the injured tissue, and
treating the injured tissue with one or more bioactive agents before, during, and/or after emitting the therapeutically effective amount of ultrasonic energy.

47. An article of manufacture for delivering ultrasound therapy, comprising:
an ultrasound therapy device for delivering ultrasonic treatment to a biological entity, comprising:
a waveform generator configured to generate a first driving signal, the first driving signal comprising at least a first waveform segment and a second waveform segment different from the first waveform segment;
one or more transducers electrically coupled to the waveform generator, the one or more transducers configured to receive the first driving signal and to generate a first ultrasonic signal based in part on the first driving signal;
one or more sensors operable to determine at least one physiological characteristic of the biological entity;
a controller electrically coupled the waveform generator and the one or more sensors, the controller configured to perform a comparison of the determined at least one physiological characteristic of the biological entity to stored reference data, and to generate a response based in part on the comparison; and
a package insert providing instructions for administering, to a subject in need of ultrasonic therapy, a therapeutically effective amount of at least one dosage form.

48. The article of manufacture of claim 47, wherein the package insert further comprises: at least one of
a table of intensity dose settings in W/cm²;
a table of lesion depth versus signal intensity settings;
a table of degree of tissue injury versus signal intensity settings;
a table of degree of tissue injury versus pulse ratio settings;
a table of size of treatment area versus duration settings;
a table of size of treatment area versus signal Intensity settings; and
a table of size of treatment area versus pulse ratio settings; or combinations thereof.
